(19) United States
(12) Patent Application Publication
(10) Pub. No.: US 2017/0354492 A1
(43) Pub. Date: Dec. 14, 2017

(54) OPHTHALMIC LENS INSERTER APPARATUS AND METHOD

(71) Applicant: Presbibio, LLC, Irvine, CA (US)

(72) Inventors: Vladimir Feingold, Irvine, CA (US); David John Smith, Highland, CA (US); Helio Enrique Gomez, South Pasadena, CA (US)

(73) Assignee: Presbibio, LLC, Irvine, CA (US)

(21) Appl. No.: 15/177,799

(22) Filed: Jun. 9, 2016

(51) Int. Cl. A61F 2/14; A61F 2/16

(52) U.S. Cl. CPC A61F 2/148; A61F 2/1453; A61F 2/167; A61F 2210/0004

(57) ABSTRACT

An embodiment in accordance with the present invention provides an apparatus and method for inserting a lens into pocket in the cornea. The apparatus includes a handle, a plunger extending movably through the lumen of the handle, and a first and a second actuators. In particular the plunger includes a distal segment which extends beyond the distal end of the handle and the first and second actuators are coupled to the plunger configured in such a way to provide different ranges of motion and synchronized movement of a bottom, middle, and top tines of the plunger for improved and safer lens insertion into the corneal pocket.

### FIELD OF THE INVENTION

The present invention relates generally to ocular surgery. More particularly, the present invention relates to an apparatus and method for inserting an intracorneal lens into a corneal pocket to treat presbyopia.

### BACKGROUND OF THE INVENTION

Presbyopia is the gradual loss of near vision, which often accompanies the aging process and which ultimately results in cataracts. The eyes of a person suffering from presbyopia have a diminished ability to focus on near objects such as books, magazines, or a computer screen. Symptoms of presbyopia can include difficulty reading fine print and blurred vision when transitioning the focus of the eye between near and distant objects.

There are several common treatments for presbyopia. A dedicated pair of reading glasses is one such treatment. Reading glasses provide magnification of near objects to provide for improved vision. However, if a person also needs glasses to focus on distant objects switching between reading glasses and distance glasses can be inconvenient. Another treatment is bifocal glasses, which provide a portion of the glasses lens for assisting with distance vision and a portion for assisting with near vision. While bifocals provide a single pair of glasses for both near and distance vision correction, they can cause disorientation. Contact lenses for the surface of the eye have also been developed which provide vision correction for both near and distance vision. Although these treatments provide vision correction for a person suffering from presbyopia, each requires at least one an additional accessory or pair of contact lenses that must be worn or used daily.

In efforts to eliminate the limitations of the aforementioned vision correction methods, very small lenses for insertion into the eye, and in particular, a corneal pocket were recently developed. These lenses however, unlike intraocular lenses which are used replace the natural lens after a patient is diagnosed with cataracts, require precise and careful handling to successfully implant the lens without damaging the person's cornea. The assignee of the present application, for example, has disclosed new devices for the insertion of these lenses in U.S. patent application Ser. No. 13/908,557 (now issued as U.S. Pat. No. 9,017,401) and Ser. No. 15/038,700 from PCT/US14/066540 (published as WO 2015/080933 A1). Those insertion devices disclosed have been able to provide precise positioning while carefully handling the lens for insertion but still can present various sterilization and misalignment challenges. Another prior art device was described in U.S. Pat. App. Pub. No. 2012/0245592 A1, assigned to Neoptics A G. This device is based on a two-part design principle used to minimize handling of the micro-lens by the practitioner prior to insertion. More particularly, the device includes a reusable handle and a pre-load one-off use unit which is used to contain the lens and push off the lens onto the pocket using a slide having a front end that complements the shape of the perimeter of the lens. This device design may compromise the optical properties of the lens when it is pushed off from one side (limiting precision and complicating the insertion procedure), and/or prevent inspection of the lens prior to implantation.

While the available prior art seeks to preserve the integrity of the lens and ease insertion, additional further improved devices are desired to overcome one or more of: (A) provide an insertion tool that does not require alignment or assembly of highly delicate parts that are critical to the success of the procedure by the medical practitioner; (B) provide an insertion tool that is completely disposable to prevent re-use of parts that may be contaminated and/or damaged; (C) provide an insertion tool that reduces inadvertent operation by mechanizing the loading and offloading of the micro-lens in a precise manner; (D) provide an insertion tool that allows inspection of a micro-lens by a physician before insertion into a patient's eye; and (E) provide an insertion tool that doesn't require the micro-lens to be pushed off a carrier and enables precision placement.

Accordingly, it is desirable to provide additional apparatus and methods for inserting a lens into the cornea to improve a patient's presbyopia.

### SUMMARY OF THE INVENTION

The foregoing needs are met, to a great extent, by the present invention, wherein in some aspects of embodiments of the invention are intended to address one or more of the above noted fundamental problems associated with devices for insertion of a lens into an eye that do not take into account the small and fragile size of intracorneal lenses and the characteristics of corneal pockets. More specifically, an improved lens inserter that can contain the lens throughout an insertion procedure for accurate placement inside a corneal pocket with minimal impact to the surrounding tissue.

According to some aspects of the disclosure, an apparatus for inserting a lens into an eye is disclosed. The apparatus for inserting a lens into an eye including a handle having a distal end and a proximal end and an outer wall of the handle defining a lumen extending through the handle; a plunger, including a top tine, a center tine, and a bottom tine, extending movably through the lumen of the handle having a distal end and a proximal end. A distal segment of the plunger extends beyond the distal end of the handle. A first actuator coupled to the plunger configured to provide horizontal sliding movement to the plunger and a second actuator configured to separate an end of the top tine from a lens supporting slot structure provided by one or both of said bottom tine and said center tine are also provided. The center tine can include a pin at an angle that causes it to protrude thru the top tine to secure the lens during insertion in a corneal pocket.

In some embodiments, a spring is disposed within the lumen of the handle against which the plunger is biased. Other springs may be included, including, for example, where at least one spring loaded decent configured to signal to a user when the horizontal sliding movement is at a pre-determined position. In addition, at least one spring disposed within the lumen and the second actuator may be included. This spring can be configured to displace, when the second actuator is disengaged, a structure that provides pressure to the top tine.

According to additional aspects of the disclosure, the plunger is configured to provide different sliding ranges of motion to the center, top, and bottom tines, via a single movement of the first actuator using mechanical features of the center, top and bottom tines. This may provide, for example, different sliding motion of the center tine in relation to a synchronized motion of the top and bottom tines when the first actuator is moved past an actuation point. In one non-limiting example, the bottom tine may move before one or both of the top and bottom tines when the first actuator is slid towards the proximate end of the handle.

Moreover, according to yet additional aspects of the disclosure, the actuation of the second actuator while the first actuator is slid towards/away from the proximate end of the handle can additionally provide articulated motion causing the center tine to remain fixed in relation to the top and bottom tines, allowing for loading of the lens onto a lens supporting structure and for positioning of the lens inside of a corneal pocket, accordingly.

There has thus been outlined, rather broadly, certain aspects of the invention in order that the detailed description thereof herein may be better understood, and in order that the present contribution to the art may be better appreciated. There are, of course, additional aspects of the invention that will be described below and which will form the subject matter of the claims appended hereto.

In this respect, before explaining at least one aspect of the invention in detail, it is to be understood that the invention is not limited in its application to the details of construction and to the arrangements of the components set forth in the following description or illustrated in the drawings. The invention is capable of aspects in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the invention. It is important, therefore, that the claims be regarded as including such equivalent constructions insofar as they do not depart from the spirit and scope of the invention.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention.

FIG. 1 illustrates a perspective view of an exemplary lens inserter apparatus 100 with relation to an eye 150 in accordance with aspects of the disclosure;

FIG. 2 illustrates a magnified view of an articulating plunger end 160 of the lens inserter apparatus 100 of FIG. 1 proximate to a corneal pocket 155 of an eye 150;

FIG. 3 illustrates an exploded perspective view of the lens inserter apparatus 100 of FIG. 1:

FIG. 4A illustrates a top view of the assembled lens inserter apparatus 100 shown in FIG. 3;

FIG. 4B illustrates a magnified top view of the articulating plunger end 160;

FIG. 4C illustrates a magnified side view of the articulating plunger end 160;

FIG. 5A illustrates a top view of an exemplary bottom tine 115 of the lens inserter apparatus 100 of FIG. 1;

FIG. 5B illustrates a side view of an exemplary bottom tine 115 of the lens inserter apparatus 100 of FIG. 1;

FIG. 5C illustrates a perspective view of an exemplary bottom tine 115 of the lens inserter apparatus 100 of FIG. 1;

FIG. 6A illustrates a top view of an exemplary center tine 110 of the lens inserter apparatus 100 of FIG. 1;

FIG. 6B illustrates a side view of the exemplary center tine 110 of the lens inserter apparatus 100 of FIG. 1;

FIG. 6C illustrates a magnified section side view of the distal end of the exemplary center tine 110 of the lens inserter apparatus 100 of FIG. 1;

FIG. 7A illustrates a top view of an exemplary top tine 105 of the lens inserter apparatus 100 of FIG. 1;

FIG. 7B illustrates side view of an exemplary top tine 105 of the lens inserter apparatus 100 of FIG. 1;

FIG. 7C illustrates a Perspective view of an exemplary top tine 105 of the lens inserter apparatus 100 of FIG. 1;

FIGS. 8A-8G illustrate configurations provided by the exemplary lens inserter apparatus of FIG. 1 for loading an intracorneal lens according to aspects of the disclosure;

FIGS. 9A-9F illustrate configurations provided by the exemplary lens inserter apparatus of FIG. 1 for inserting the intracorneal lens into the corneal pocket according to aspects of the disclosure; and

FIG. 11 illustrates a lens transfer tool 1100 which may be implemented with some embodiments according to the present disclosure.

The present invention is further described in the detailed description that follows.

### DETAILED DESCRIPTION OF THE INVENTION

The invention will now be described with reference to the drawing figures, in which like reference numerals refer to like parts throughout. Going forward, various aspects of the steering device of the present disclose may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present.

Relative terms such as “lower” or “bottom” and “upper” or “top” may be used herein to describe one element's relationship to another element illustrated in the drawings. It will be understood that relative terms are intended to encompass different orientations of steering device in addition to the orientation depicted in the drawings. By way of example, if aspects of steering device shown in the drawings are turned over, elements described as being on the “bottom” side of the other elements would then be oriented on the “top” side of the other elements. The term “bottom” can therefore encompass both an orientation, of “bottom” and “top” depending on the particular orientation of the apparatus.

Various aspects of the steering device may be illustrated with reference to one or more exemplary embodiments. As used herein, the term “exemplary” means “serving as an example, instance, or illustration,” and should not necessarily be construed as preferred or advantageous over other embodiments of a steering arm disclosed herein.

### Glossary

In this description and claims directed to the disclosure, various terms may be used for which the following definitions will apply:

“Articulated motion”, as used herein, can refer to the different tine parts of the lens inserter device that allow sliding motion up to one or more pre-configured ranges via the first and sometimes second actuator before one or both a positioning signal is provided to the user and/or a rate or degree of movement of one or more part(s) becomes limited in relation to the others. The signal, for example, may include a click for the user to engage/disengage an actuator depending on the position of the device pieces. In some embodiments, the articulated motion can be used to assist the practitioner in the procedure by preventing unintended movement of device parts depending on the procedural stage.

“Lens” or “Intracorneal lens”, as used herein, can refer to a biocompatible optical correction device that can be placed inside a corneal pocket of an eye. Typically, the lens can include a micro-lens having a small diameter of, for example, 1 mm-7 mm, and more specifically, preferably a diameter of 2.5 mm-3.5 mm. The lens may include a small generally central hole that can be used to fasten and handle the lens to a tool structure during insertion, facilitate precise positioning of the lens in a corneal pocket, and, in some embodiments, to provide vision correction properties to the user after insertion.

“Lens supporting structure”, as used herein, can refer to the structure formed between the bottom tine, and in some embodiments center tine, of a lens inserter device that is used to support a lens during insertion and positioning inside a corneal pocket. In some embodiments, a top tine can be included to further protect top of the lens, and thus the optical properties of the lens during an insertion into a corneal pocket, without damaging the patient's cornea.

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as one skilled in the art would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure, which is defined solely by the appended claims and applicable law. Moreover, it is noted that like reference numerals represent similar parts throughout the several views of the drawings.

The ophthalmic lens inserter and method can help enable precise insertion of an intracorneal lens into a pocket in the cornea without compromising the patient's cornea nor optical corrective properties of the lens during insertion. The corneal pocket can preferably be created by a femtosecond laser or any other precision surgical laser, such as, those used in Lasik™ surgery procedures. According to some aspects, the lens inserter includes a handle and an articulating plunger extending movably through the lumen of the handle. The articulating plunger includes a distal segment that extends beyond the distal end of the handle via at least one actuator coupled to the plunger and configured to provide predetermined movement that can facilitate placement of the lens on a lens supporting structure and insertion and placement of the lens into the corneal pocket. According to other aspects, the lens inserter is made to be disposable by including a limited number of parts and, in some embodiments, having only those parts that are inserted into the cornea and critical to the procedure of a surgical quality steel or include a coating. In some embodiments, for example, the surgical steel parts can be treated, and/or include a coating, to include anti-microbial nano-surface properties. In yet additional embodiments, the part(s) may alternatively or additionally be made to be hydrophobic and/or to have a lower friction coefficient to prevent the tool from sticking and damaging the cornea, for example. In addition, by having the entire lens inserter be disposable, not only can components be made at a lower cost, but also, by enabling disposable lens inserters using, plastics, such as those widely known epoxy compounds, coatings, and the like, the resulting inserter can be lightweight, include personalized features, and eliminate risk of misalignment of parts during sterilization.

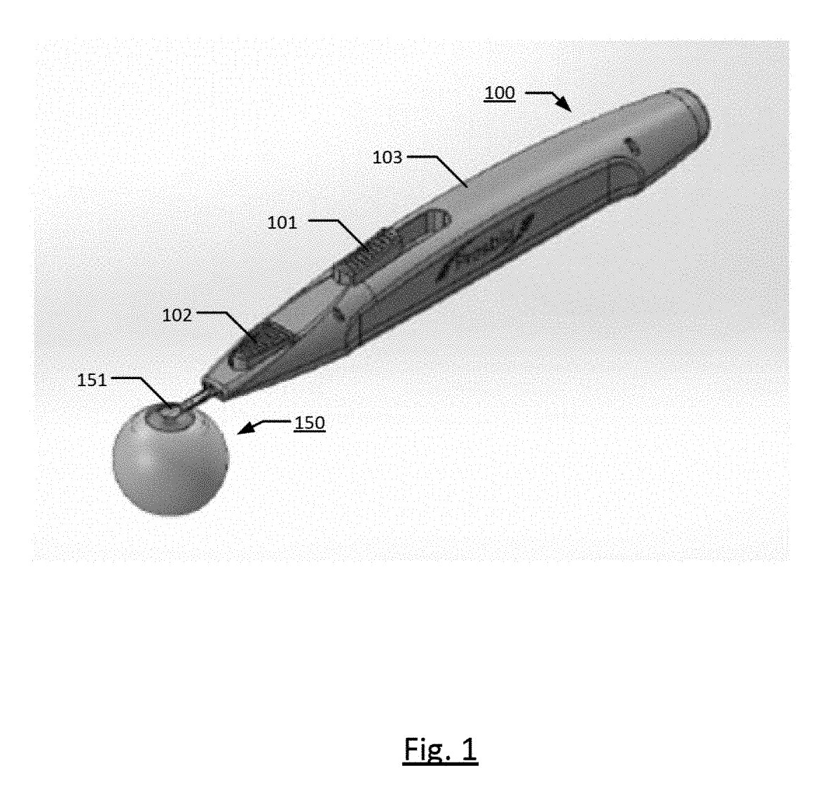

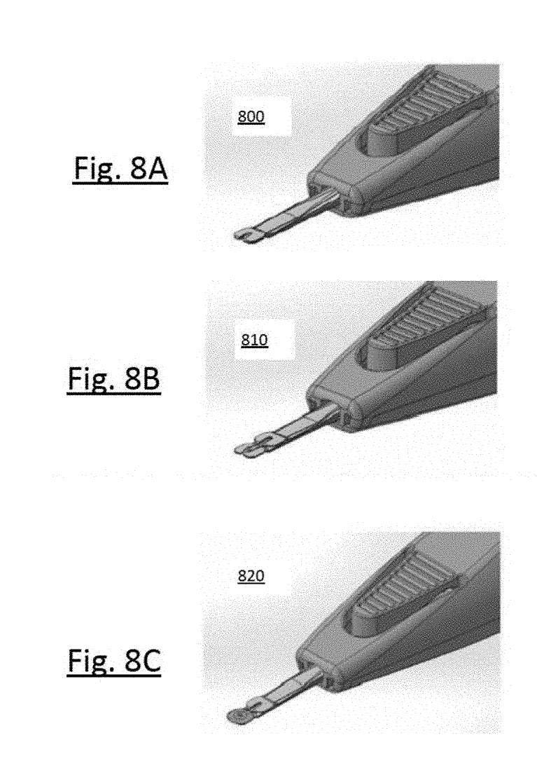

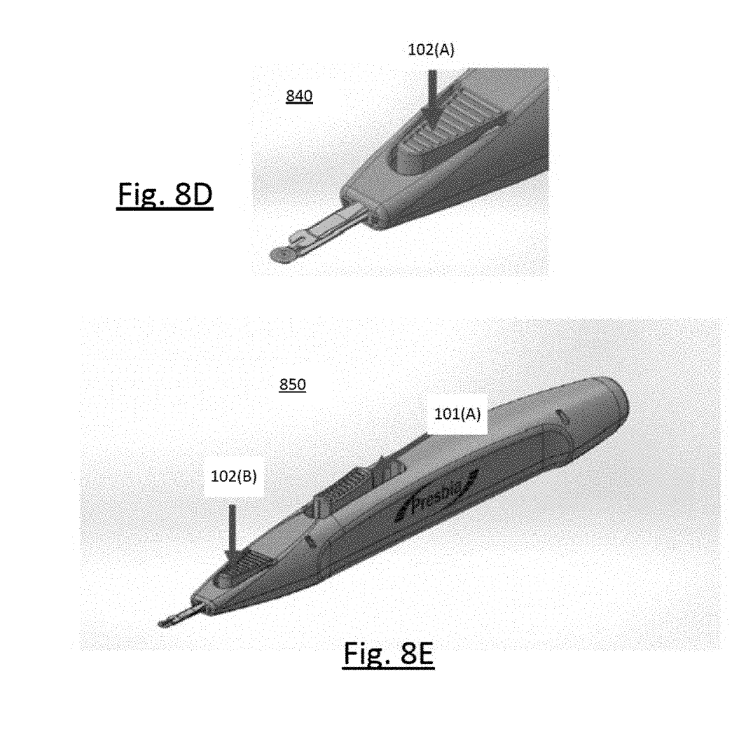

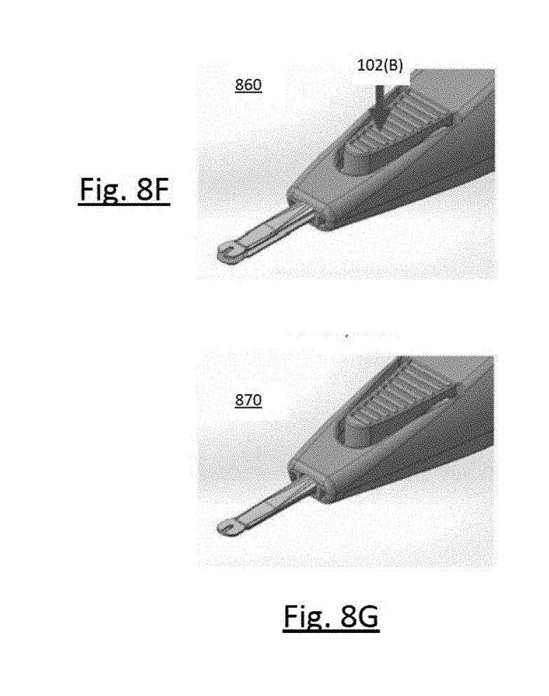

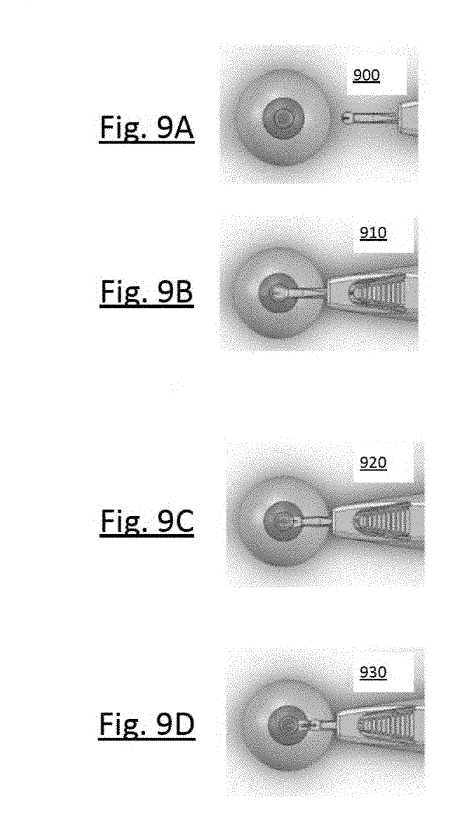

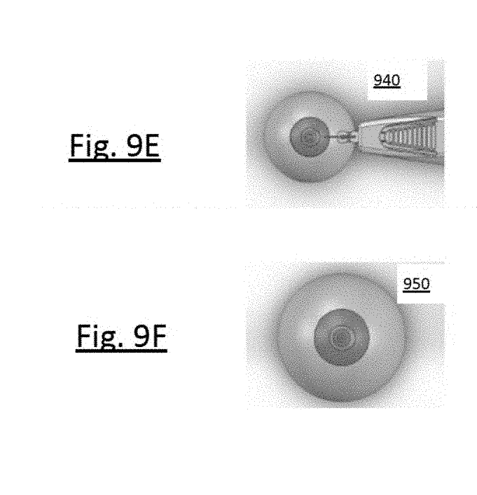

Referring now to FIG. 1, FIG. 1 illustrates a perspective view of an exemplary lens inserter apparatus 100 with relation to an eye 150 in accordance with aspects of the disclosure. In particular, the exemplary lens inserter apparatus 100 being a one-time disposable and integrated applicator that can facilitate use according to aspects of the corresponding lens insertion methods described herein. The disposable lens inserter 100 includes a housing 103 enclosing articulating parts functioning via manual operation of one or both a slidable thumb rest 101 and a lens loading actuator 102. According to some aspects, the manual operation of the slidable thumb rest 101 and lens loading actuator 102 which can enable, as further described below in the description of FIGS. 8A-8G and 9A-9F, precise and practical insertion of a lens into the cornea 151 of a patient's eye 150 to correct presbyopia related vision deficiencies.

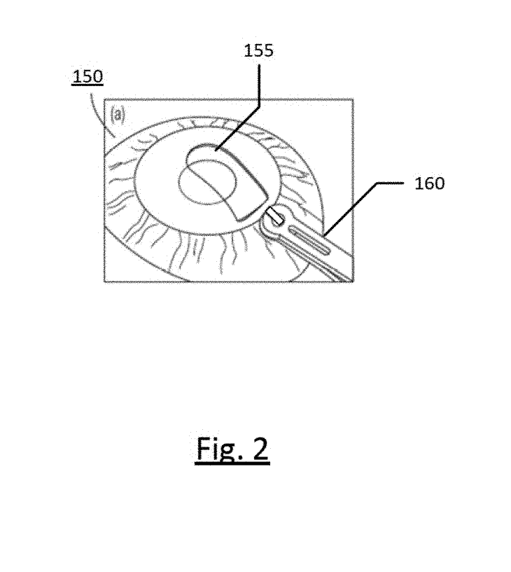

Referring now to FIG. 2, a magnified section view of an articulating plunger end 160 of the lens inserter apparatus 100 of FIG. 1 proximate to a corneal pocket 155 the an eye 150 is depicted. In particular, the section depicts an exemplary corneal pocket 155 made in the patient's eye 150 in which the microlens is inserted and positioned to provide vision correction. According to some aspects, the articulating plunger end 160 generally being contained, at least in part, inside the housing 103 and being of a surgical quality material can be used to provide support and protection to the lens and its optical properties during insertion and positioning. As previously mentioned, in some embodiments, the parts forming the articulating plunger end 160 may be surgical steel parts with treated surfaces, and/or coated, to include, for example, anti-microbial nano-surface properties, provide low friction coefficients to preserve the integrity of the cornea during insertion of the lens, and/or be hydrophobic to facilitate release of the lens after insertion. Other aspects of the disclosure relating to the structure and operation of the articulating plunger end 160 to be described in relation to the description of the proceeding figures.

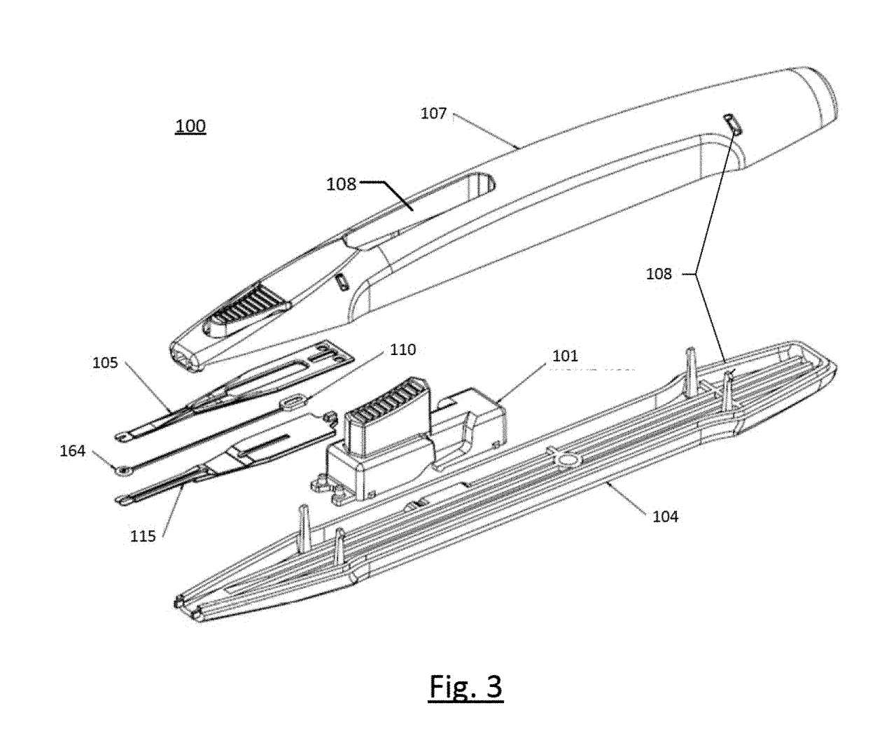

Moving on to FIG. 3, an exploded perspective view of the lens inserter apparatus of FIG. 1 is depicted. In particular, this figure demonstrates how the different parts are grated and contained by the housing 103 to operate according to aspects of the disclosure. Housing 103 may be formed by a top housing 107 and a bottom housing 104. In some embodiments, the top housing 107 and bottom housing 104 can contain complementary locking structures 109 to fix each other to the other and enclose the disposable lens inserter components. Moreover, the housing 103 may have an ergonomic shape that allows easy handling of the disposable lens inserter 100. According to some aspects the housing 103 can include a thumb rest access opening 108 that provides the practitioner to manually operate the slidable thumb rest 101. In some embodiments, the housing 103 may additionally include a lens loading actuator 102 which can apply pressure to different sections and/or parts of the articulating plunger end 160 during the loading or off-loading of the intracorneal lens 165.

According to yet additional aspects, operation of the articulating plunger end 160 can be a function of the internal structure of the housing 103 and a top tine 105, center tine 110 and bottom tine 115 moving in relation to each other in predetermined manners. For example, by each of the top tine 105, center tine 110 and bottom tine 115 attaching to the slidable thumb rest 101, the different parts can move, in a range limited by their own structural design and that of the housing 103, to extend out of a distal end of the disposable lens inserter 100. Structural features of the top tine 105, center tine 110 and bottom tine 115, can include, for example, various channels for them to integrate one to another in a pre-determined range of lateral motion, depressions to allow the loading lens actuator to separate or unite the top tine 105 and the bottom tine 115 from one another, stoppers and other locking mechanisms, variations in thickness and/or materials, and the such.

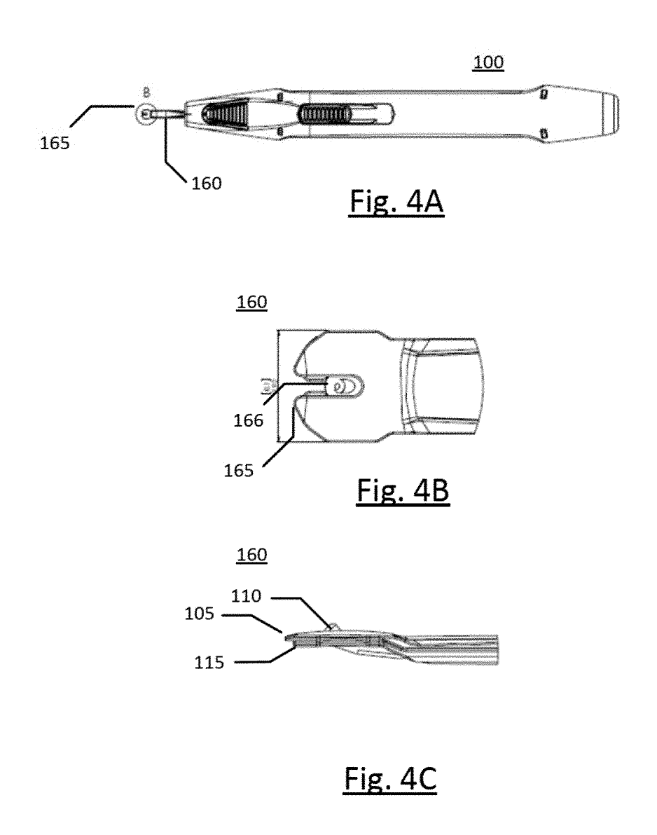

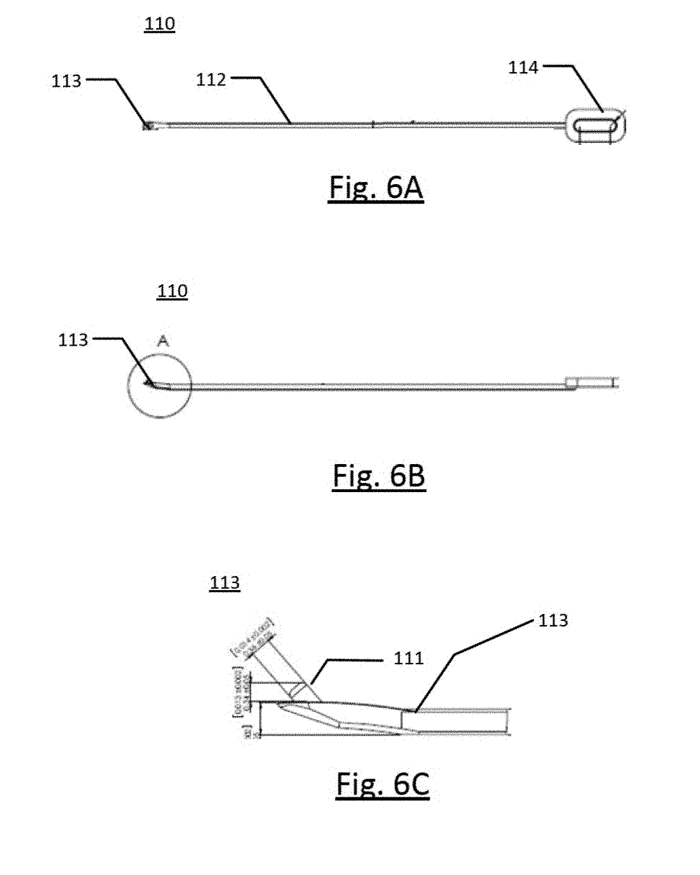

The assembled aforementioned parts of the disposable lens inserter 100 can be shown in FIG. 4A which illustrates a top view of the assembled lens inserter apparatus 100 of FIG. 3. In particular, FIG. 4 shown the slidable thumb rest 101 slid forward causing the articulating plunger end 160, shown holding an intracorneal lens 165, to protrude out of the housing 103. FIGS. 4B and 4C show a magnified top and side view section of the articulating, plunger end 160. In particular, the magnified top views showing how the intracorneal lens 165 may be contained between the distal ends of the top tine 105 and bottom tine 115. According to some aspects of the disclosure, an end of the center tine 110 can have an obtuse angle for it to extend upwards and through a section of the top tine 105 to pass thru and secure thereto the intracorneal lens 165 until it is inserted into the corneal pocket 155. By securing the intracorneal lens 165 through its central hole, the risk of affecting optical properties in the peripheral region of the lens are significantly reduced. (The center tine structure is further explained in detail in relation to FIGS. 6A-6C.) In some embodiments, the top and bottom surfaces of the intracorneal lens 165 are protected by the leaf-like ends of bottom tine 115 and top tine 105. In addition or alternatively, in some embodiments, the bottom tine 115 may provide additional support to the intracorneal lens 165 in addition to the center tine 110.

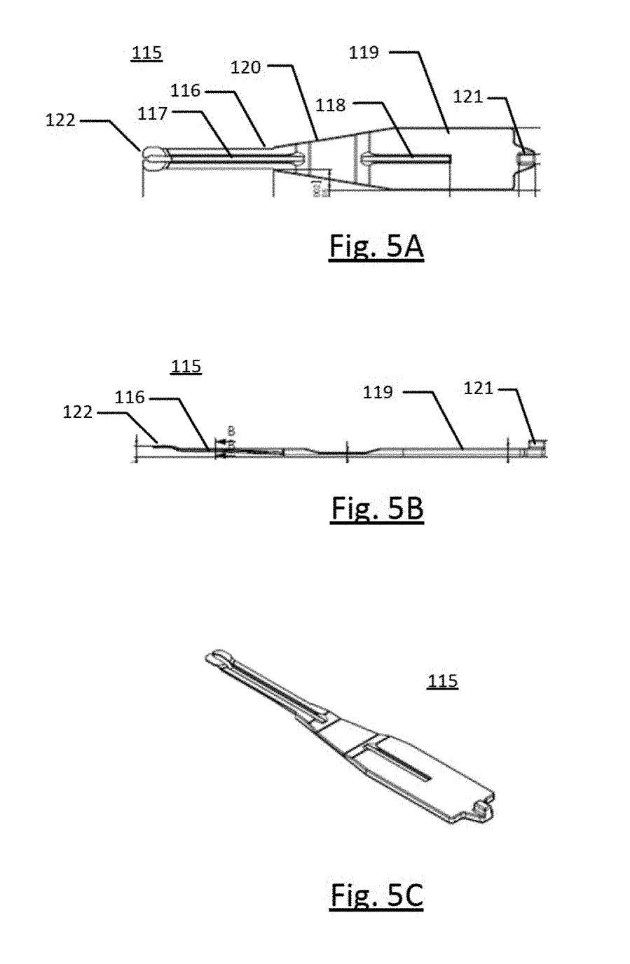

Referring now to FIGS. 5A-5C, top, side, and perspective views, respectively, of an exemplary bottom tine 115 are shown. In particular, the bottom tine 115 is shown having a distal end 116 including an elongated channel 117, for the center tine 110 to horizontally traverse therein, and connecting the bottom tine body 119 to the distal bottom leaf-like end 122. In some embodiments, the distal bottom leaf-like end 122 is configured to support the periphery of the intracorneal lens' bottom surface. As shown, in some embodiments, the bottom tine body 119 can include a bottom tine body's elongated channel 118 that allows only the center tine 110 to slide therein according to a predetermined range of motion.

In accordance with some aspects of the disclosure, the bottom tine 115 can include articulating structural features and attaching features, such as articulating structure 120 and bottom tine slider fastening structure 121, for controlled movement. For example, in the present embodiment, bottom tine slider fastening structure 121 can be used to attach a corresponding portion 114 (shown in FIGS. 6A-6C) of the center tine 110 thereto and/or to the slidable thumb rest 101. Moreover, the articulating structure 120 can be used to limit movement in relation to the other center 110 and top 105 tines and/or to allow some flexibility to take place when the lens loading actuator 102 is pressed on.

Referring now to FIGS. 6A-6C, top and side views, and a magnified section of the side view, respectively, of the exemplary center tine 110 of the lens inserter apparatus 100 of FIG. 1 are shown. In particular, the center tine 110 including an intracorneal lens engaging structure 111 located in the center tine's distal end 113 that is at an obtuse angle in relation to the center tine body 112. In some embodiments, the intracorneal lens engaging structure 111 is configured to be at an obtuse angle that is greater than 30 degrees and less than about 90 degrees, such that at least a portion of it may protrude thru a central hole of the intracorneal lens 165. In some embodiments, the lens engaging structure 111 may be generally flexible (e.g. rubber-like structure) or detachably removable to provide for safety precautionary options. In yet additional embodiments, the lens engaging structure 111 may be made of a biodegradable and biocompatible composition and/or be made to rotate about a hinge attached to the center tine. According to some aspects of the disclosure, by having the lens engaging structure 111 provide support from the central hole of the intracorneal lens 165, the intracorneal lens 165 can be sufficiently engaged thereto upon loading of it and during insertion and alignment inside the corneal pocket 155, minimizing the risk of compromising the structural optical qualities of the intracorneal lens 165. As previously mentioned, the center tine 110 can also include a center tine slider fastening structure 114 to attach to one or more of a corresponding structure of the bottom tine 121, structure of top tine 126, and the slidable thumb rest 101.

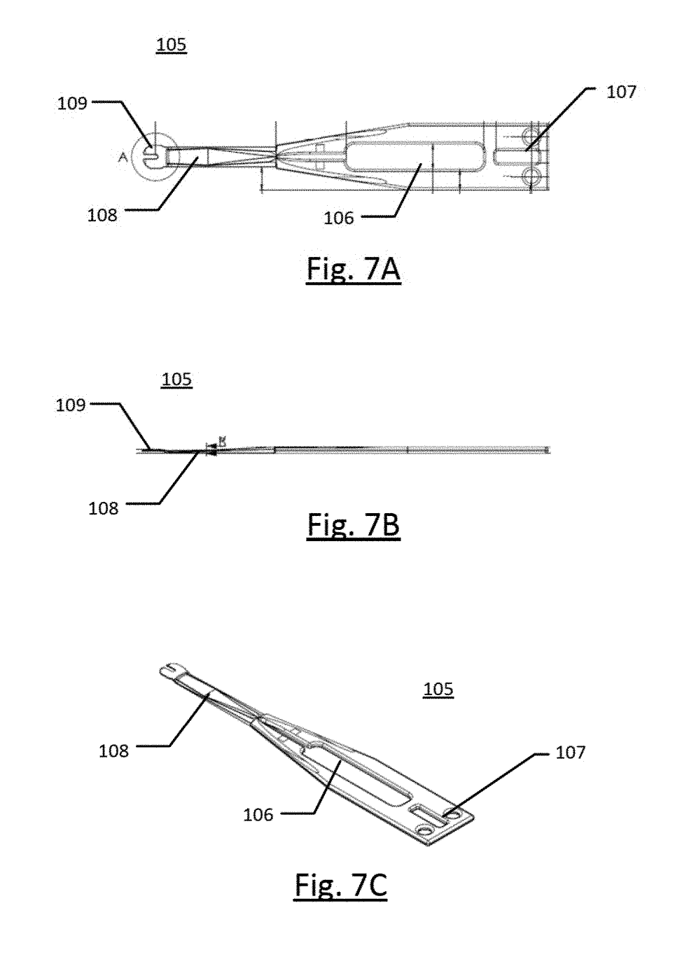

Referring now to FIGS. 7A-7C, top, side, and perspective views, respectively, of the exemplary top tine 105 are shown. In particular, the top tine 105 including a 106 top tine sliding structural feature 106 on the top tine body 125. The top tine body 125 leading towards a distal end 123 having a top leaf-like structure for protecting the top surface of the intracorneal lens 165. In some embodiments, the top line body 125 can include a top tine articulating feature 124 which may be pressed on by the lens loading actuator 102, for example, to cause the top leaf-like structure to move away from the intracorneal lens 165 and/or one or both of the center tine 110 and the bottom tine 115 when loading or positioning the intraocular lens 165. The top tine 105 may additionally be fastened to one or both of the center 110 and bottom 115 tines and the slidable thumb rest 101. With respect to the structural aspects of the tines 105, 110, and 115 and the housing 103, one skilled in the art will understand from the disclosure that articulated motion of one or more parts may be encouraged or limited by one or more spring loaded parts, detent pins, hand retractable, pins, buspring loaded pins, bspring loaded pins, button handle pins, shackle pins, and the such, such that the disposable lens inserter may function according to the particular aspects of the disclosure and, particularly, as described in FIGS. 8A-8G during the loading of the intracorneal lens onto the disposable lens inserter, and FIGS. 9A-9F during the insertion of the intracorneal lens into a corneal pocket, as to prevent damage to the patient's cornea 151, risk of infection from the procedure, and/or compromising the optical characteristics of the intracorneal lens.

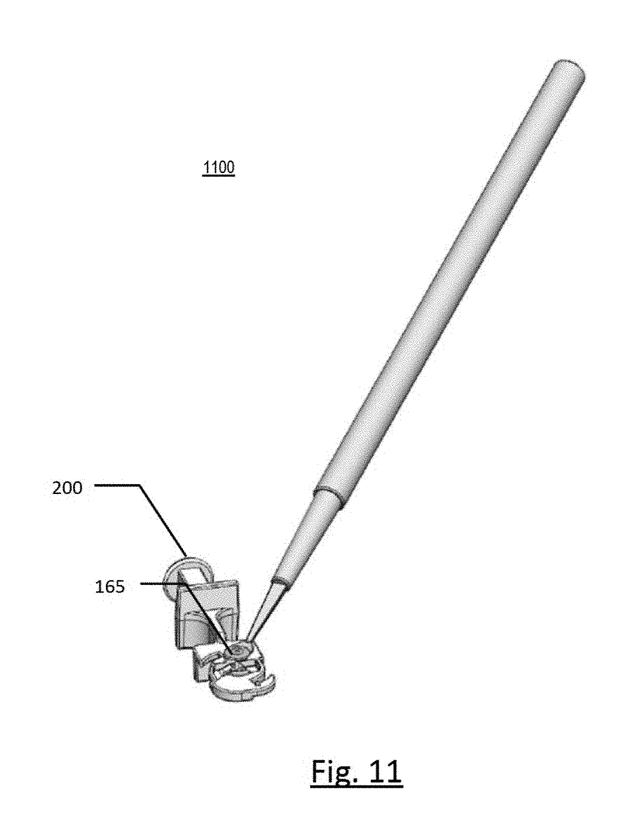

Referring back to FIGS. 8A-8G, disposable lens inserter 100 configurations 800-870 according to aspects enabled by the disposable lens inserter that may be used for loading an intracorneal lens onto a lens supporting structure. In particular, at 800 the articulating plunger end 160 of a disposable lens inserter is shown when the slidable thumb rest 101 is pushed forward by a user in anticipation of loading the intracorneal lens. Once the thumb rest 101 reaches a predetermined position for lens loading, the thumb rest 101 can click into a position in which the top tine is pulled back, for example, via some spring loaded detents, to achieve the loading position shown at 810. At 820, the intracorneal can be loaded onto a lens engaging feature of the top tine by using a separate transfer tool, such as the transfer tool illustrated in FIG. 11 at 1100, which may be used to carefully handle and transport the intracorneal lens 165 from its package 200. At 840, the lens loading actuator 102 can be pushed in the direction shown by 102(A). While holding down the lens loading actuator 102, the slidable thumb rest 101 can be pushed forward in the direction of 101(A) to begin sliding the top tine forward as shown at 850. Continuing to press the lens loading actuator 102, once the slidable thumb rest 101 is pushed all the way forward, as shown at 860, the top tine leaf-like structure can cover the top surface of the intracorneal lens, which may again cause the slidable thumb rest 101 to click, indicating to the user that the lens loading actuator 102 can be depressed. Once the lens loading actuator 102 is depressed, the leaf-like structure moves toward the lens as shown at 870, to decrease the width that must be inserted into the corneal pocket 155. Maintaining the position shown at 870, the intracorneal lens can be in a position in the disposable lens inserter for insertion into the corneal pocket 155.

FIGS. 9A-9F illustrate configurations 900-950 according to aspects enabled by the disposable lens inserter that may, be used for inserting and aligning the loaded intracorneal lens in a corneal pocket 155. In particular, at 900 the disposable lens inserter 100 can be manually handled by a practitioner and guided towards the corneal pocket 155 until at least a portion of the articulating plunger end containing the intracorneal lens is inserted into the corneal pocket 155, as shown in 910. Once the articulating plunger end is about a position in which the intracorneal lens 165 is to be located, at 920, the slidable thumb rest 101 can be pulled back such that the top tine can move away up to a predetermined distance away from the top surface of the intracorneal lens and imparting very minimal force due in part to the structural flexibility (to keep it from causing injury to the cornea). Continuing to slide the thumb rest 101, at 930, the top and bottom tines can retract away from the corneal pocket and back into the disposable inserter device 100. Once the slidable thumb rest 101 is pushed back all the way, the top and bottom tines are fully retracted, as shown at 940, only the center tine is holding the intracorneal lens allowing for improved visibility and manipulation, thus ultimately more precise alignment. Once the intracorneal lens 165 is aligned, the lens inserter may be removed easily, leaving the lens inside the pocket and aligned, as shown at 950, due to the obtuse angle of the lens engaging feature and the structural flexibility that the center tine may have.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

We claim:

1. An apparatus for inserting a lens into an eye comprising:
a handle having a distal end and a proximal end and an outer wall of the handle defining a lumen extending through the handle;
a plunger, including a top tine, a center tine having a lens engaging structure, and a bottom tine, extending movably through the lumen of the handle having a distal end and a proximal end, wherein a distal segment of the plunger extends beyond the distal end of the handle;
a first actuator coupled to the plunger configured to provide horizontal sliding movement to the plunger; and
a second actuator configured to separate an end of the top tine from a lens supporting slot structure provided by one or both of said bottom tine and said center tine, and
wherein the lens engaging structure arises from the center tine at an obtuse angle such that it protrudes thru one or both of a hole of the lens and an opening of the top tine.

2. The apparatus of claim 1, wherein the lens engaging structure and the top tine are configured to secure the lens during insertion in a corneal pocket.

3. The apparatus of claim 1, additionally comprising:
a spring disposed within the lumen of the handle against which the plunger is biased.

4. The apparatus of claim 1, additionally comprising:
at least one spring loaded detent configured to signal to a user when the horizontal sliding movement is at a pre-determined position.

5. The apparatus of claim 1, additionally comprising:
at least one spring disposed within the lumen and the second actuator, the spring being configured to displace, when the second actuator is disengaged, a structure that provides pressure to the top tine.

6. The apparatus of claim 1, wherein the engaging structure of the center tine is removably fixed to the center tine.

7. The apparatus of claim 6, wherein the removably fixed engaging structure is made of a biodegradable and biocompatible composition.

8. The apparatus of claim 1, wherein the engaging structure can rotate about a hinge attached to the center tine.

9. The apparatus of claim 1, wherein the plunger is configured to provide different sliding ranges of motion to the center, top, and bottom tines, via a single movement of the first actuator.

10. The apparatus of claim 9, wherein the sliding motion of the center tine is different to a synchronized motion of the top and bottom tines when the first actuator is moved past an actuation point.

11. The apparatus of claim 9, wherein the bottom tine moves before one or both the top and bottom tines when the first actuator is slid towards the proximate end of the handle.

12. The apparatus of claim 11, wherein the center tine moves last in relation to the top and bottom tines when the first actuator is slid towards the proximate end of the handle.

13. The apparatus of claim 9, wherein the actuation of the second actuator while the first actuator is slid towards the proximate end of the handle causes the center tine to remain fixed in relation to the top and bottom tines, allowing for positioning of the lens inside of a corneal pocket.

14. An apparatus for inserting a lens into an eye comprising:
a handle having a distal end and a proximal end and an outer wall of the handle defining a lumen extending through the handle;
a plunger, including a top tine, a center tine, and a bottom tine, extending movably through the lumen of the handle having a distal end and a proximal end, wherein a distal segment of the plunger extends beyond the distal end of the handle;
a first actuator coupled to the plunger configured to provide horizontal sliding movement to the plunger;
a second actuator coupled to the plunger configured to separate an end of the top tine from a lens supporting slot structure provided by the distal ends of one or both of said bottom tine and said center tine; and
wherein the bottom, center and top tines of the plunger provide different sliding ranges of motion controlled via the actuation of the first and a second actuator.

15. The apparatus of claim 14, wherein the pin and the top tine are configured to secure the lens during insertion in a corneal pocket.

16. The apparatus of claim 14, wherein the actuation of the second actuator while the first actuator is slid towards the proximate end of the handle causes the center tine to remain fixed in relation to the top and bottom tines to allow for positioning of the lens inside of a corneal pocket.

17. The apparatus of claim 15, wherein the sliding motion of the center tine is different to a synchronized motion of the top and bottom tines when the first actuator is moved past an actuation point.

18. An apparatus for inserting a lens into an eye comprising:
a handle having a distal end and a proximal end and an outer wall of the handle defining a lumen extending through the handle;
a plunger, including a top tine, a center tine, and a bottom tine, extending movably through the lumen of the handle having a distal end and a proximal end, wherein a distal segment of the plunger extends beyond the distal end of the handle;
a first actuator coupled to the plunger configured to provide horizontal sliding movement to the plunger;
a second actuator coupled to the plunger configured to separate an end of the top tine from a lens supporting slot structure provided by the distal ends of one or both of said bottom tine and said center tine; and
wherein the bottom, center, and top tines are synchronized to move at different positions of the first actuator.

19. The apparatus of claim 18, wherein the pin and the top tine are configured to secure the lens during insertion in a corneal pocket.

20. The apparatus of claim 18, wherein the sliding motion of the center tine is different in relation to the movement of the top and bottom tines depending on the direction on which the first actuator is slid towards.
